(19) United States
(12) Patent Application Publication
(10) Pub. No.: US 2026/0289782 A1
(43) Pub. Date: Sep. 24, 2026

(54) SYSTEM AND METHOD FOR DETECTING AND REPORTING ERRORS IN DIGITAL MICROFLUIDIC EXPERIMENTS

(71) Applicant: NUCLERA LTD, Cambridge (GB)

(72) Inventor: Sepehr JALALI, Cambridge (GB)

(21) Appl. No.: 19/475,591

(22) PCT Filed: Apr. 19, 2024
(86) PCT No.: PCT/GB2024/051022

§ 371 Date: Oct. 15, 2025

(30) Foreign Application Priority Data
Apr. 19, 2023 (GB) 2305756.5

(51) Int. Cl. G06T 7/00; G06T 5/80; G06T 7/33; G06T 7/73; G06V 10/25; G06V 10/26; G06V 10/75; G06V 10/764; G06V 10/82

(52) U.S. Cl. CPC G06T 7/0014; G06T 5/80; G06T 7/337; G06T 7/74; G06V 10/25; G06V 10/26; G06V 10/751; G06V 10/764; G06V 10/82; G06T 2207/20084; G06T 2207/30168; G06T 2207/30204

(57) ABSTRACT

A method for detecting and reporting errors in a digital microfluidic experiment (20), the method comprising: receiving image data comprising an image (160) of a digital microfluidic volume; detecting one or more features of interest (170) in the image; determining a spatial distribution and shape of the detected one or more features of interest; receiving an expected spatial distribution and shape (150) of features; comparing the determined spatial distribution or shape of the detected one or more features of interest with the received expected spatial distribution or shape of features; determining one or more erroneous features of interest based on the comparison; and reporting the presence or absence of one or more erroneous features of interest.

### FIELD OF INVENTION

The present invention relates to a system and method for detecting and reporting errors in digital microfluidic experiments involving droplet manipulation.

### BACKGROUND OF THE INVENTION

In digital microfluidic experiments, droplets of fluid confined in an enclosed volume can be manipulated based on electrowetting using arrays of electrodes. In known experiments, such electrodes can take the form of a thin film transistor electrode array. Manipulating the droplets can include moving them, splitting them and merging them with other droplets. This enables a range of experiments to be designed using such systems.

Digital microfluidic experiments can be designed using software configured to simulate the digital microfluidic environment. Using this software, an operator can outline how each droplet of fluid should be manipulated as the experiment progresses. The result is a sequence of commands that the operator can run on the device. When executed by the digital microfluidic device, the device is instructed to apply an electric potential to move droplets in a manner that should, in theory, manipulate the droplets in the way the operator has designed.

However, errors in the experiment may occur. For example, droplets may merge with others unexpectedly, causing contamination; droplets may not move to their expected position; droplets may split in a way that is undesirable; or droplets may be larger or smaller than expected. This list of possible errors is non-exhaustive. In addition images of droplets may show objects that look like droplets but are in fact contaminants, either within the device enclosure, such as air bubbles, or external to the enclosure such as dust or scratches on the device surface. There is a need for a way to detect such errors so that they can be accounted for, ignored or corrected, or the experiments repeated as required.

WO 2019/227126 A1 describes a system for controlling microfluidic droplets or bubbles. The system performs droplet tracking using a Hough transform to detect the circular boundary of droplets. Unlike the present invention, there is no comparison of the droplet tracking result (using the Hough transform) with an expected spatial distribution to determine erroneous features. In addition, the use of the Hough transform limits the kinds of errors that can be detected compared to the present invention. WO 2019/227126 A1 is only concerned with detecting single vs multiple droplets, whereas the comparison with expected spatial distribution of the present invention allows erroneous positions, relative positions, sizes, shapes of droplets, as well as missing features to be detected.

WO 2019/153067 A1 describes a method for detecting and reporting errors in a digital microfluidic experiment using an image-based feedback system. The image-based feedback system uses a Hough transform to detect circular features within a user-defined box. Again unlike the present invention, there is no comparison of the Hough transform result with an expected spatial distribution to determine erroneous features. In addition, the use of the Hough transform limits the kinds of errors that can be detected compared to the present invention. In particular, it is not possible using the Hough transform method of WO 2019/153067 A1 to detect missing features, or extraneous features, among other error types made possible via the comparison between detected and expected spatial distribution as per the present invention.

Willsey et al “Scaling Microfluidics to Complex Dynamic Protocols”, 2019 IEEE International Conference on Computer-Aided Design (ICCAD) describes a runtime system providing a high-level API for microfluidic manipulations. Willsey et al specifies that droplets are detected by dying them with green dye, and then calibrating the vision system to that hue. The use of a dye to detect droplets limits the kinds of errors that can be detected using the system since only dyed droplets are detected by the system, and not unwanted air bubbles for example. Willsey et al also does not disclose a comparison between a detected and expected spatial distribution of features.

LUO et al “Error Recovery in Cyberphysical Digital Microfluidic Biochips”, IEEE Transactions on Computer-Aided Design of Integrated Circuits and Systems, vol. 32, 1 Jan. 2013, New York, USA describes error detection in a microfluidic system. The system uses a template-matching algorithm to detect droplets. There is no comparison between a detected spatial distribution and an expected spatial distribution to detect errors, the only comparison is against what a particular single droplet should look like.

US 2022/372468 A1 describes an error detection method for microfluidics experiments. Again, there is no comparison of expected vs detected spatial distributions, and US 2022/372468 A1 uses fluorescence in order to detect droplets, thus the system could not detect extraneous features, or unwanted air bubbles for example, and therefore the types of errors that can be captured are limited.

Alistar et al “Redundancy optimization for error recovery in digital microfluidic biochips”, Design Automation for Embedded Systems, vol. 19, 13 Jan. 2015, New York, USA describes a method for error detection in a digital microfluidic experiment. Alistar et al only describes measuring droplet volumes to determine the correct size of droplets. There is no disclosure of a comparison between an expected and detected spatial distribution to determine erroneous features in the experiment.

### SUMMARY OF THE INVENTION

The invention is defined in the independent claims below to which reference should now be made. Optional features are set forth in the dependent claims.

An example method is described in more detail below and takes the form of a method for detecting and reporting errors in a digital microfluidic experiment. The method comprises receiving image data comprising an image of a digital microfluidic volume; detecting one or more features of interest in the image; and determining a spatial distribution and shape of the detected one or more features of interest. The method further comprises receiving an expected spatial distribution and shape of features and comparing the determined spatial distribution and shape of the detected one or more features of interest with the received expected spatial distribution and shape of features. The presence or absence of one or more erroneous features of interest based on the comparison are determined. The presence or absence of one or more erroneous features is subsequently reported.

The inventors have appreciated that the progress of a digital microfluidic experiment can be monitored by capturing images of the digital microfluidic volume. Errors in the experiment can, therefore, be detected by using image processing methods to compare what is being shown by the image of the experiment to what is expected based on a simulation of the experiment.

According to an aspect of the present disclosure, there is provided a method for detecting and reporting errors in a digital microfluidic experiment, the method comprising: receiving image data comprising an image of a digital microfluidic volume; detecting one or more features of interest in the image; determining a spatial distribution and shape of the detected one or more features of interest; receiving an expected spatial distribution and shape of features; comparing the determined spatial distribution and shape of the detected one or more features of interest with the received expected spatial distribution and shape of features; determining one or more erroneous features of interest based on the comparison; and reporting the presence or absence of one or more erroneous features of interest.

The detected one or more features of interest in the image may correspond to droplets of fluid in the digital microfluidic volume. The detected one or more features of interest in the image may correspond to pellets of beads in the digital microfluidic volume.

The detecting one or more features of interest in the image may be performed using a trained convolutional neural network.

The method may further comprise training a convolutional neural network to detect features of interest using images of digital microfluidic volumes in which features of interest have been segmented.

The method may further comprise prior to the detecting one or more features of interest, adjusting the received image.

Adjusting the received image may comprise applying a transformation to the received image to correct a lens distortion, the applying a transformation comprising applying a lens distortion matrix.

The method may further comprise prior to the transforming the received image, calculating the lens distortion matrix.

Adjusting the received image may comprise enhancing the visibility of the features of interest in the image of the digital microfluidic volume.

Comparing the determined spatial distribution of the detected one or more features of interest with the expected spatial distribution of features may comprise: spatially aligning the received image and the expected spatial distribution and shape of features; and mapping features of interest in the image to corresponding features in the expected spatial distribution.

Spatially aligning the received image and the expected spatial distribution of features may comprise: detecting a plurality of fiducial markers in the received image; and applying a transformation to the received image based on the detected fiducial markers.

Detecting one or more erroneous features of interest may comprise determining that a position or shape of a detected feature of interest in the received image does not match a position or shape of a corresponding feature of interest the expected spatial distribution, or vice versa.

Detecting one or more erroneous features of interest may comprise determining that the size and shape of a detected feature of interest in the received image does not match a size and shape of a corresponding feature of interest in the expected spatial distribution.

Detecting one or more erroneous features of interest may comprise detecting a feature of interest in the received image with no corresponding feature of interest in the expected spatial distribution, or vice versa.

The expected spatial distribution may correspond to a simulation of the digital microfluidic volume and no errors are reported.

Determining the spatial distribution of the detected one or more features of interest may comprise generating bounding boxes around each of the detected one or more features of interest. Where the features are for example round or irregular in shape, the determination may involve the use of contours. The features of interest may comprise only a part of a fluidic volume, for example a protrusion that loads last. If the analysis shows the protrusion fails to load, the droplets can be marked as failed or of concern.

Comparing the spatial distribution of the detected one or more features of interest with the expected spatial distribution may comprise comparing a spatial distribution of the generated bounding boxes with the expected spatial distribution.

The method may further comprise assigning a category to a detected erroneous feature of interest based on the comparison of the determined spatial distribution of the detected one or more features of interest with the received expected spatial distribution of features. The category may comprise a size mismatch category, and/or one of: a missed object category and an extra object category.

The method may further comprise annotating the received image based on the detected one or more erroneous features of interest.

The method may further comprise acting to correct the erroneous features. The method may further comprise knowing that a particular feature will give rise to an error, and either ignoring data generated by that particular feature or acting to remove said feature from further operations.

According to an aspect of the present disclosure, there is provided an error detection and reporting system for a digital microfluidic volume, the system comprising: processing means configured to: receive image data comprising an image of a digital microfluidic volume; detect one or more features of interest in the image; determine a spatial distribution and shape of the detected one or more features of interest; receive an expected spatial distribution and shape of features; compare the determined spatial distribution and shape of the detected one or more features of interest with the received expected spatial distribution and shape of features; detect the presence or absence of one or more erroneous features of interest based on the comparison; and report the presence or absence of one or more erroneous features of interest.

The system may comprise a digital microfluidic volume configured to manipulate droplets of fluid. The digital microfluidic volume may comprise one or more fiducial markers configured to enable mapping features of interest in the image to corresponding features in the expected spatial distribution.

The correctly shaped droplets may be square or rectangular in shape and the incorrectly shaped droplets may be irregular in shape, indicating an incorrect volume of fluid in the droplets. Monitoring may be performed by matching the observed shape to the desired shape in order to identify anomalies.

The system may comprise an illumination means and an imaging means configured to capture images of the digital microfluidic volume and provide image data comprising the captured images to the processing means. The imaging means may be a camera.

Disclosed herein is a non-transitory computer-readable medium on which are encoded instructions that, when executed cause a processing means to perform a method comprising: receiving image data comprising an image of a digital microfluidic volume; detecting one or more features of interest in the image; determining a spatial distribution and shape of the detected one or more features of interest; receiving an expected spatial distribution and shape of features; comparing the determined spatial distribution and shape of the detected one or more features of interest with the received expected spatial distribution and shape of features; determining the presence or absence of one or more erroneous features of interest based on the comparison; and reporting the presence or absence of one or more erroneous features of interest.

### BRIEF DESCRIPTION OF DRAWINGS

The invention will be described in more detail below by way of examples with reference to the accompanying drawings, in which:

FIG. 1 is a block diagram of an example system according to aspects of the present disclosure;

FIG. 2 is a flowchart of an example method according to aspects of the present disclosure;

FIGS. 3A and 3B are respectively an example of an expected spatial distribution and shape and its corresponding image of a digital microfluidic experiment according to aspects of the present disclosure;

FIG. 4 is an example of an expected spatial distribution and shape of features according to aspects of the present disclosure;

FIGS. 5A and 5B are respectively an example of an image of a digital microfluidic experiment before and after pre-processing according to aspects of the present disclosure;

FIGS. 6A and 6B are respectively a second example of an image of a digital microfluidic experiment before and after pre-processing according to aspects of the present disclosure;

FIG. 7 is an example of an image of a digital microfluidic experiment with features of interest identified in bounding boxes according to aspects of the present disclosure;

FIGS. 8A, 8B and 8C are an example of increasingly zoomed in images of a digital microfluidic volume according to aspects of the present disclosure;

FIG. 9 is an example image of a digital microfluidic volume overlaid with indicators for spatial alignment according to aspects of the present disclosure;

FIG. 10 shows an example image of a digital microfluidic experiment (left hand side) and the corresponding manually annotated image (right hand side) according to aspects of the present disclosure;

FIGS. 11A and 11B are respectively an example of an image of a digital microfluidic experiment before and after features of interest have been segmented according to aspects of the present disclosure;

FIG. 12 is an example of an image of a digital microfluidic experiment in which features of interest have been assigned categories according to aspects of the present disclosure;

FIGS. 13A and 13B are respectively an example of an image of a digital microfluidic volume and a corresponding 2D binary mask of the detected spatial distribution according to aspects of the present disclosure; and

FIG. 14 is an example of an image of a digital microfluidic experiment in which bounding boxes identify features of interest and a region of interest has been selected according to aspects of the present disclosure.

### DETAILED DESCRIPTION

### Digital Microfluidic Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrokinesis occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semiconductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c \cdot {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWoD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/ε)^{1/2}. Thus, to reduce actuation voltage, it is required to reduce (t/ε)^{1/2} (i.e., increase dielectric constant or decrease insulator/dielectric thickness).

To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100 V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWoD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminium oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick. The conformal layer may be between 100 nm and 200 nm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers. The method is particularly suitable for aqueous droplets with a volume of 1 μL or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference.

Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
   a second substrate comprising a top electrode;
a spacer disposed between the first substrate and the second substrate and defining an electrokinetic workspace; and
   a voltage source operatively coupled to the matrix electrodes;

Described herein is an electrokinetic device, including:
a first substrate having a matrix of electrodes, wherein each of the matrix electrodes is coupled to a thin film transistor, and wherein the matrix electrodes are overcoated with a functional coating comprising:
one or more dielectric layer(s) comprising silicon nitride, hafnium oxide or aluminum oxide in contact with the matrix electrodes,
a conformal layer comprising parylene in contact with the dielectric layer, and
a hydrophobic layer in contact with the conformal layer;
a second substrate comprising a top electrode;
a spacer disposed between the first substrate and the second substrate and defining an electrokinetic workspace; and
a voltage source operatively coupled to the matrix electrodes;

The electrokinetic devices as described may be used with other elements, such as for example devices for heating and cooling the device or reagent cartridges for the introduction of reagents as needed.

The device can be an active-matrix thin film transistor (AM-TFT) based device.

Also disclosed is an active-matrix thin film transistor (AM-TFT) device having a substrate bearing a plurality of electrodes, the device comprising multiple fluidic inlet ports on at least two sides of the device, wherein the inlet ports on each side of the device are evenly spaced and wherein the device is connected to a syringe pump.

The device may comprise two substrates, wherein at least one substrate has a plurality of electrodes, and the two substrates define parallel plates that are separated by a spacer to define a volume.

The fluidic entry may come via holes in the upper plate or through the spacer. The entry holes may be in the top substrate. The plurality of electrodes may be on a bottom substrate. The top substrate be of glass or polymer and may have a thickness ranging from 0.5 mm to 20 mm.

The spacer may comprise an adhesive with beads of a defined size distribution. The spacer may comprise a polymer material of a defined thickness. The spacer may comprise glass, in which case the layers can be fused together. The spacer gap and therefore height of fluid in the device may be between 50 microns and 250 microns. The spacer gap and therefore height of fluid in the device may be between 100 microns and 150 microns.

The enclosed volume may contain aqueous droplets in a hydrophobic filler fluid. The filler liquid may be a hydrophobic or non-ionic liquid. For example the filler liquid may be decane or dodecane. The filler fluid may be a silicone oil such as dodecamethylpentasiloxane (DMPS). The filler liquid may contain a surfactant, for example a sorbitan ester such as Span 85.

The filler liquid may be moved via an automated manner, or may be moved under gravity. A hydrostatic head of pressure can be used to move the filler liquid within the device. The aqueous phase may be loaded via wells on the device. The wells may be at least partially filled with filler fluid before the aqueous reagents are loaded. The filler fluid may be less dense than the aqueous phase such that the aqueous phase sinks in the wells.

The device may be connected to a pump, for example a syringe pump, a peristaltic pump, a disc pump, a diaphragm pump, or a pneumatic pump. The pump enables filling of the device with filler liquid in an automated manner. Once filled, the pump enables partial withdrawal of the filler fluid to create a negative pressure in the device which draws in reagents from the wells. Thus the filling and withdrawal of fluid may be performed in an automated manner to allow largely ‘hands-free’ loading of the aqueous reagents. An automated filler liquid filling and withdrawal method may be integrated into an instrument that provides other functions relating to the digital microfluidic device, including heating, cooling, optical, sensing, mechanical, and magnetic functions.

The wells of the device may be at 90 degrees to each other. The inlets may be at 180 degrees to each other. The inlets may be on 4 sides of the device. Each side may have at least 4, 8 or 12 ports. Each side may have 8 ports. The device may have 4 sets of 8 ports. The number of ports may vary on different sides of the device, for example one side may have 8 ports and one side 4 ports. The device may have 8 ports on 3 sides and 16 ports on a fourth side. The device may have 16 ports on 3 sides and 8 ports on a fourth side. The ports may be offset to give multiple rows of linear ports on one side, for example a first and second row where the second row is behind by offset from the first row such that the source liquid can flow between the ports of the first row. The rows may be a zig-zag fashion.

The pitch between inlet ports may be 9 mm. The pitch between inlet ports may be 4.5 mm. The inlet ports have a pitch of 4.5 mm or a multiple of thereof. This would cover 24 well, 48 well, 96 well, 384 well ports. The pitch of the ports may be the same on each side of the device, or may be different sized. In this context the pitch refers to the distance between the centre of each inlet.

The volume of aqueous reagents loaded per inlet port may be between 1 microlitre and 50 microlitres. The volume may be between 1 microlitre and 20 microlitres.

The aqueous liquid may be introduced to the wells by a pipette, a multichannel pipette, a syringe, a blister pack, an acoustic dispenser, or a robotic liquid handler. The aqueous liquids may be loaded simultaneously from multiple wells, which may be on the same side or multiple sides of the devices. Each well is a separate liquid, and can be the same or different to the contents of the aqueous volume in other wells. The volume of aqueous liquid loaded in each port can be the same or can be different.

The automated filling and/or withdrawing of filler fluid may be controlled by software. The device may be part of a larger instrument system that provides environmental control such as temperature control or light control and may have analytical capabilities such as optical systems for fluorescence or luminescence assay detection.

The location of the aqueous layer is controlled by the actuation of electrodes to form reservoirs in defined areas. A plurality of electrodes is actuated to control the location of the aqueous liquid once it has been drawn onto the substrate bearing a plurality of electrodes.

Multiple reservoirs may be formed on the device.

### Specific Embodiment

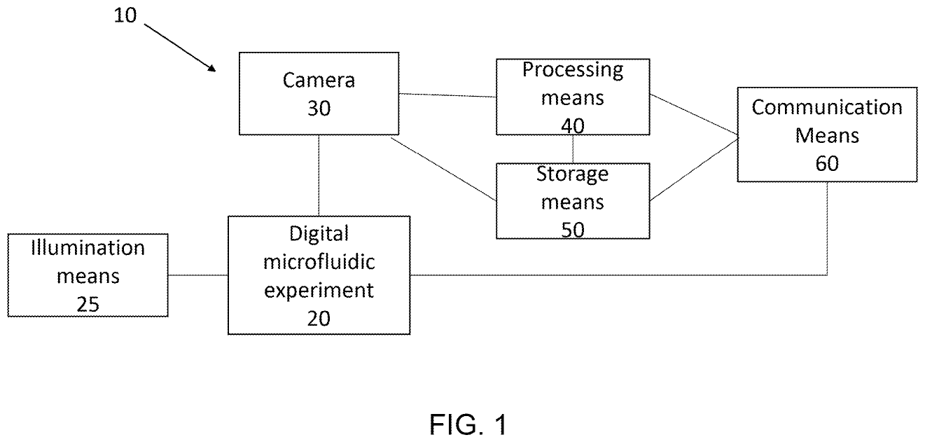

An example of a system and method for detecting and reporting errors in a digital microfluidic experiment is described in detail below with reference to FIGS. 1 to 14. Like features are given like reference numerals.

An example system 10 for detecting and reporting errors in a digital microfluidic experiment is illustrated by the block diagram of FIG. 1. The system 10 comprises a digital microfluidic experiment 20. The system further comprises illumination means 25. The illumination means 25 is configured to illuminate the digital microfluidic experiment for subsequent imaging. In this example, the illumination means is configured to illuminate the digital microfluidic experiment with light of a first wavelength. The illumination means may be further configured to illuminate the digital microfluidic experiment with light of a second wavelength. More specifically, the first illumination corresponds to white light. The second illumination may correspond to blue or ultraviolet (UV) light. If the droplets of fluid comprise fluorescent proteins, blue or UV light makes the droplets fluoresce, allowing the density of proteins in a given droplet to be measured. The white light illuminates the digital microfluidic experiment volume and aids in tracking the droplets.

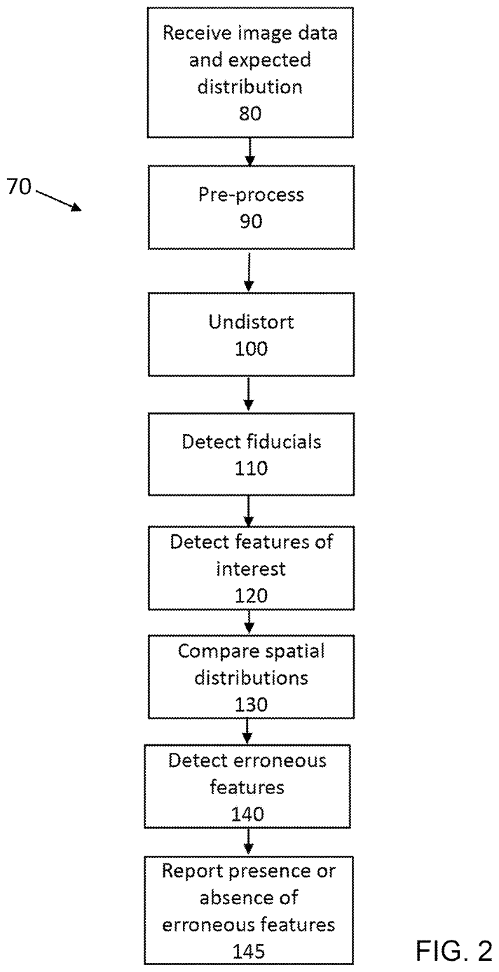

The system 10 further comprises imaging means 30. In this example, the imaging means is a camera 30. The camera 30 is configured to capture images or videos of the digital microfluidic experiment 20. More specifically, the camera 30 is configured to capture images of the digital microfluidic volume (not shown). The camera 30 is further configured to provide image data comprising the captured images to processing means 40. The processing means 40 is configured to execute the method outlined in the flowchart of FIG. 2. This feature is significant and is discussed in more detail below. The system 10 further comprises storage means 50. Said storage means 50 is configured to store data. Said data includes one or more of image data captured by the camera 30, data processed by the processing means 40, and data regarding the set-up and execution of the digital microfluidic experiment 20 based on the experiment the operator has designed. The system 10 further comprises communication means 60. Said communication means 60 is configured to exchange data between the experiment 20 and cloud storage holding data regarding the set-up and execution of digital microfluidic experiments.

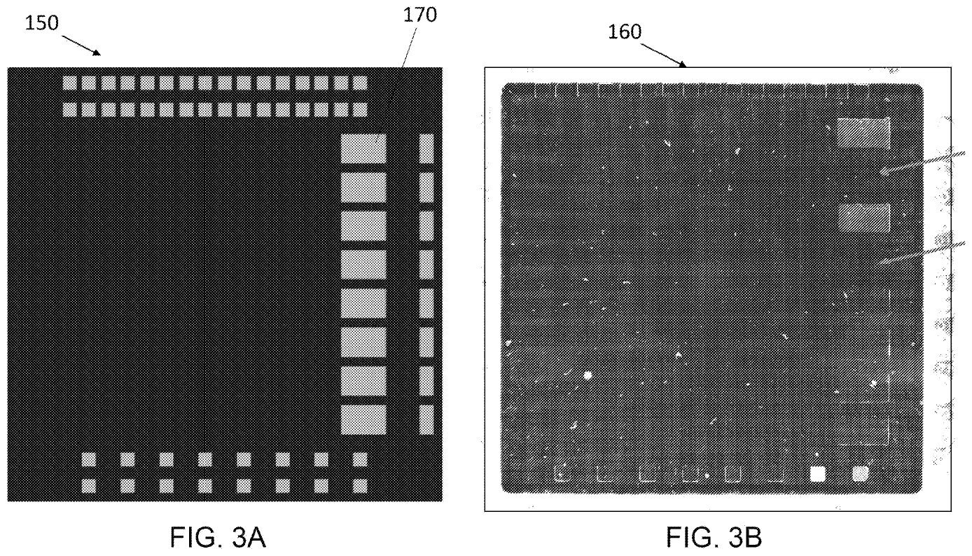

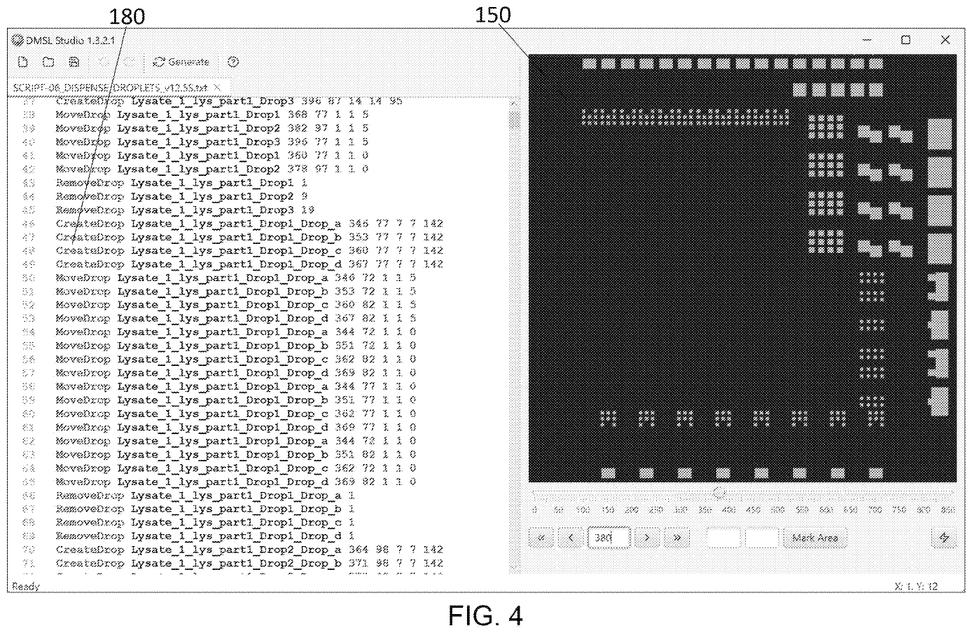

As explained above, the processing means 40 is configured to implement the method outlined in the flowchart 70 of FIG. 2. The method outlined detects errors in a digital microfluidic experiment 20 from images captured by the camera 30. Once the camera 30 has captured an image of the digital microfluidic volume, image data comprising said image is received by the processing means 40 at a first step 80. The processing means 40 is further configured to receive data corresponding to an expected spatial distribution and shape of features. In this example, the expected distribution corresponds to a simulation of the digital microfluidic experiment 20. FIG. 3A depicts a 2D representation of an expected spatial distribution and shape 150 and FIG. 3B depicts the corresponding image 160 taken of the actual experiment simulated by FIG. 3A. The features 170 in the simulation 150 indicate where spatially in the digital microfluidic volume droplets are expected to be located at a point in time during the experiment. The features 170 further indicate the expected size and shape of the droplets at a given point in time. The expected spatial distribution and shape 150 may be described by a script 180 such as that shown in FIG. 4. The script 180 corresponds to the simulation 150 of the digital microfluidic experiment. Corresponding features as seen in the simulation 150 should be detectable in the image 160 of the digital microfluidic volume. If this is not the case, there may be an error in the experiment. The system 10 described herein aims to detect such errors. The expected spatial distribution may be represented as shown in FIG. 3A, but may be stored as, and received as, a list of expected features (that is, features that are expected to be present within the experiment volume at a given point in time), and their respective properties (size, shape, position, etc.). For example, the expected spatial distribution may be stored as an M×N matrix, where M represents the number of expected features in the experiment volume at a given time and N represents the number of characteristics stored for each feature. It will be appreciated that the number of expected features (i.e. droplets) in the experiment volume can change throughout the duration of an experiment. Accordingly, there may be multiple expected spatial distributions stored for a given experiment, as many as one per time increment of the experiment. Alternatively, the expected spatial distribution may be stored as an M×N×T matrix, where T is the number of time increments of the experiment. The expected spatial distribution may be stored in memory as a JSON (JavaScript Object Notation) file, or other suitable file type. The expected spatial distribution may be stored alongside a list of fiducial marker positions and active and ignore areas. These features are described in more detail below.

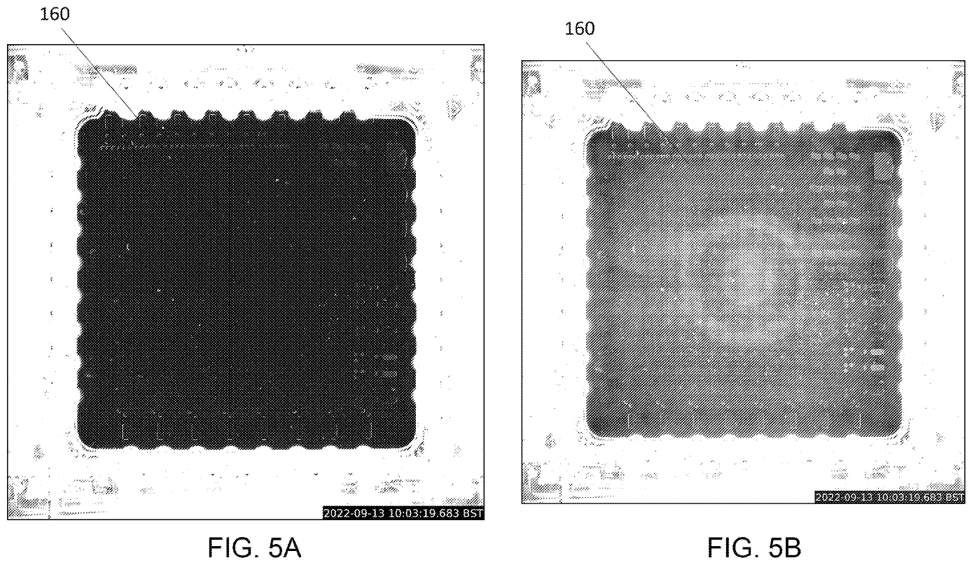

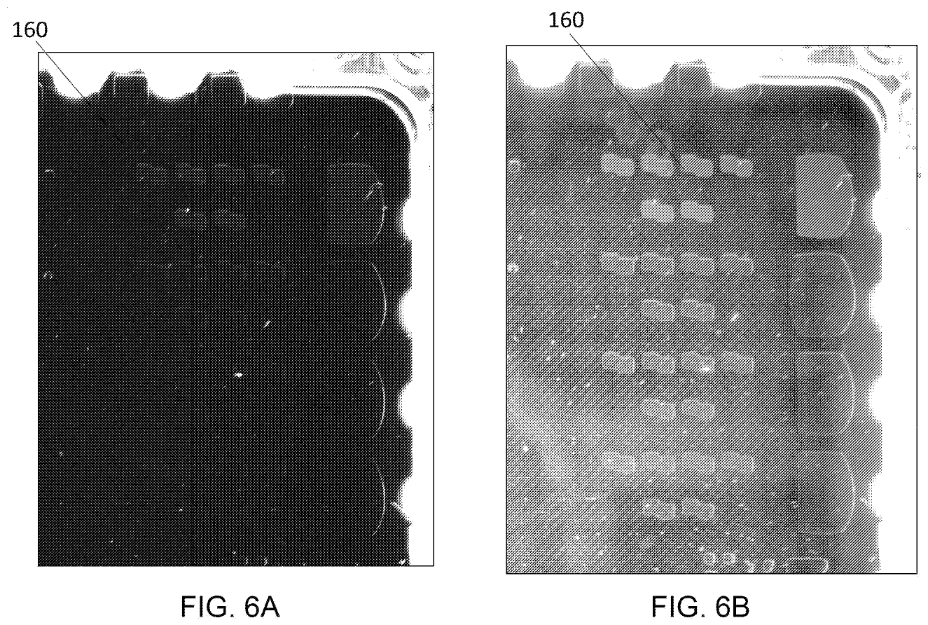

Once the image data is received, the image 160 may be adjusted. Image adjustment is done before any features of interest are detected in the image. In this example, adjusting the image 160 comprises a pre-processing step 90. Specifically, pre-processing the image 160 to enhance the visibility of features of interest in the image 160. This is seen by comparing the initial image 160 of FIG. 5A with the image 160 of 5B which has undergone such a pre-processing step 90. A second example is depicted by FIGS. 6A and 6B This makes said features easier to detect in following stages of processing. Numerous image processing techniques for enhancing visibility of features of interest in an image are known to the skilled person, for example contrast limited adaptive histogram equalization (CLAHE). CLAHE is a variant of adaptive histogram equalization that limits contrast amplification to reduce noise amplification by performing histogram equalization in small patches or small tiles with high accuracy and contrast limiting. In this example, said pre-processing step 90 includes one or more of increasing contrast, increasing brightness or applying a filter to the image 160.

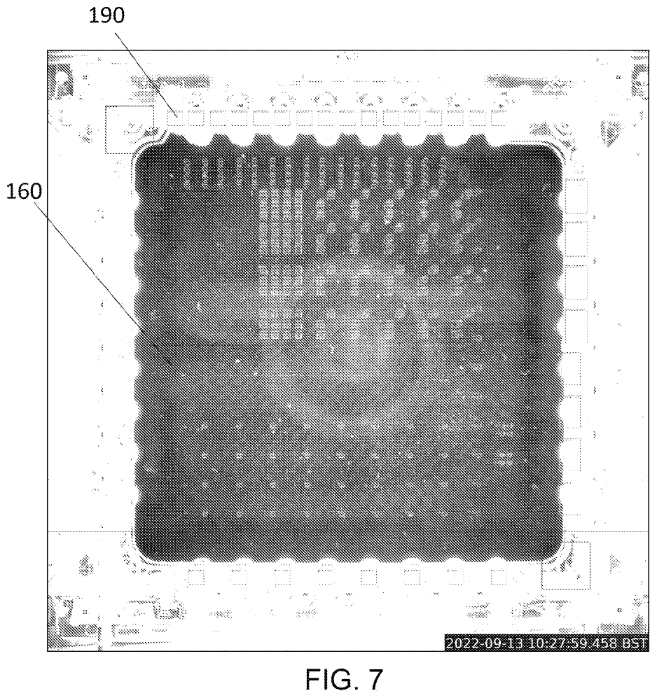

Adjusting the image 160 further comprises correcting for lens distortion. Lens distortion is a result of the physical properties of the imaging means 30 used to capture the image. For example, the angle at which the camera 30 captures the image of the digital microfluidic volume. Imperfections in the curvature(s) of one or more lenses used to focus the light onto the detector of the imaging means 30 can cause various types of lens distortion. Lens distortion can result in warping of the projection of the digital microfluidic volume that is captured by the image 160. In other words, if the digital microfluidic volume is a perfect square, this would not translate to a perfect square in the image 160 (i.e. by pixel number) as a result of lens distortion. For features of interest in the image 160 to be mapped to corresponding features in the expected distribution 150, such distortion needs to be accounted for. In other words, mapping between features is not linear. Furthermore, camera optics introduce radial distortion in the image 160 of the digital microfluidic volume. Radial distortion is the result of pixel density being higher in the centre of the image 160 and lowering radially toward the edges. In other words, inaccuracy in mapping between a radially distorted image 160 and an expected distribution 150 increases moving radially away from the centre of the image 160. Such inaccuracy is present in the image of FIG. 7 in which bounding boxes 190 indicate the expected spatial distribution and shape 150 of features. Said bounding boxes 190 match actual features in the image 160 more closely in the centre of the image than at the edges.

To account for this, correcting for lens distortion comprises applying a transformation to the image. In this example, the transformation comprises a lens distortion matrix. The matrix is applied to every image 160 of a digital microfluidic volume taken by the camera. The matrix corrects for the above described distortion resulting from the imaging means 30. In other words, applying the matrix to a captured image undistorts the image. However, as the way in which an image 160 is distorted is unique to the camera 30 itself and its set-up (e.g. camera angle), a new matrix is calculated every time the camera 30 is adjusted or a new camera is used to implement the method described herein. Calculation of a new lens distortion may be performed using a calibration technique, for example by imaging a predetermined calibration pattern and comparing the detected image with the expected pattern geometry. The predetermined calibration pattern be a grid pattern, checkerboard pattern, symmetric circle grid pattern, asymmetric circle grid pattern, or any other suitable calibration pattern. For example a checkerboard or dot-grid can be used to find distortion in images in each instrument. Once an instrument is calibrated, the parameters are stored and used for any images taken with that instrument later on.

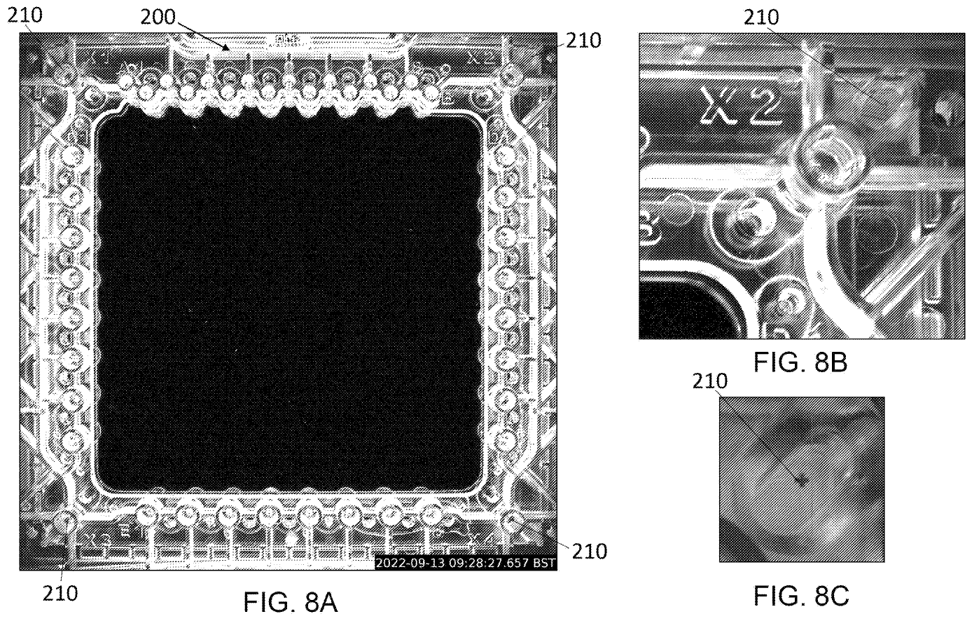

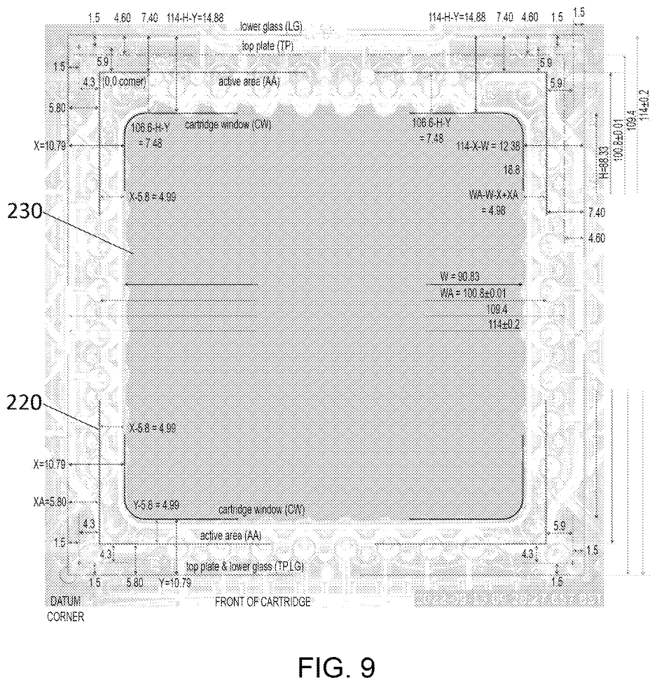

To ensure accurate mapping between features of interest in the image 160 and the expected spatial distribution and shape 150, at a further step 110 the image 160 of the digital microfluidic volume and the expected spatial distribution and shape 150 are spatially aligned. Such alignment ensures that the image 160 of the digital microfluidic volume spatially corresponds to the simulated volume 150. In this example, the digital microfluidic volume 200 comprises fiducial markers 210 to enable alignment. Specifically, each corner of the volume 200 comprises a marker 210, as shown in FIGS. 8A, 8B and 8C. At this step 110 of the method, the markers 210 are detected in the image 160 of the digital microfluidic volume 200. The markers 210 act as reference points for subsequent alignment as shown in FIG. 9. In other words, a transformation is applied to the image 160 based on the detected fiducial markers 210. Said transformation enables alignment with the simulation 150 of the volume 200. Using the markers 210, regions such as the active area 220 and the cartridge window 230 can be delimited for comparison with the simulated volume 150. This can be thought of as a calibration step.

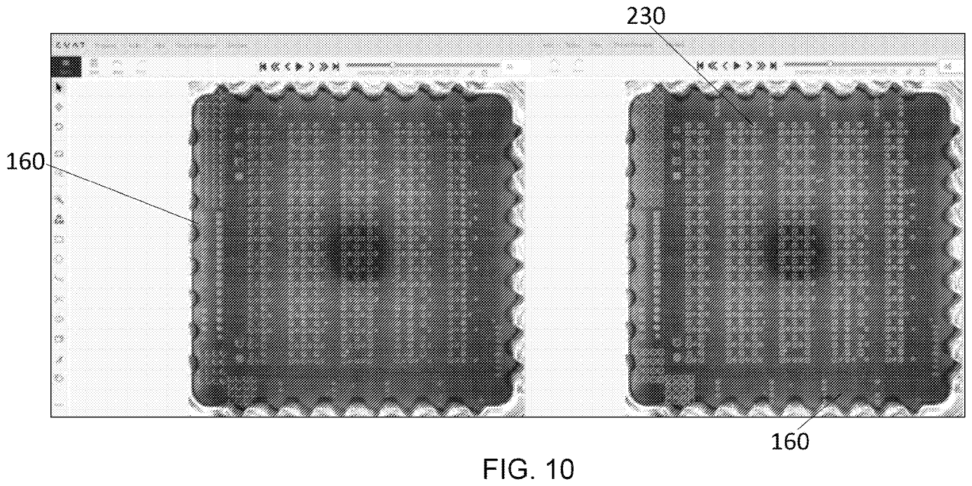

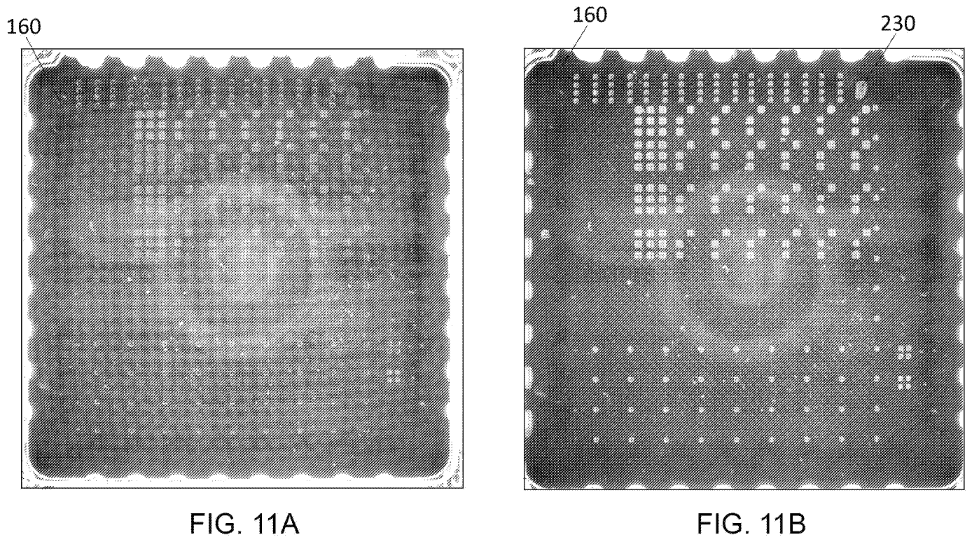

At step 120 of the method outlined by the flowchart 70 of FIG. 2, features of interest are detected in the image 160 of the digital microfluidic volume 200. Machine and deep learning methods are employed to do so. Specifically, a trained convolutional neural network is used to detect features of interest in the image 160. In this example, the convolutional neural network used is U-Net, as described in RONNEBERGER, O et al. “U-Net: Convolutional Networks for Biomedical Image Segmentation”. arXiv. doi 10.48550/ARXIV.1505.04597. 2015. U-Net is a known deep learning framework developed for image segmentation. Specifically, image segmentation in the biomedical field. U-Net is known to be accurate, making it ideal for the purposes described herein. The neural network is trained before use in the method outlined by the flowchart 70 of FIG. 2. Training of neural networks is known to the skilled person, with various possible approaches. The term “training” is known in the art, and refers to the process of adjusting one or more parameters of a model in order to improve the model performance. An example of a well-known algorithm for training a neural network is the backpropagation gradient descent algorithm. The neural network is trained using annotated images of digital microfluidic experiments. In these images, features of interest are segmented such that the network learns to identify such features. FIG. 10 shows an example image 160 of a digital microfluidic experiment (left hand side) and the corresponding annotated image (right hand side). In this example, the image is annotated manually to segment features of interest 230. More specifically, the image 160 is annotated using Computer Vision Annotation Tool (CVAT). CVAT is a known platform for labelling/annotating training data. Other such tools will be known to the person skilled in the art. In FIG. 10, features of interest have been identified. Once trained, the model can identify and segment features of interest in subsequent images 160 of digital microfluidic experiments received at step 80 of the method described by flowchart 70 of FIG. 2. The trained model detects and outputs contours of objects in the image 160, and each contour may be used to determine a bounding box around the object in the image. An example of this is shown in FIGS. 11A and 11B. The features of interest 230 in the image 160 of 11A have been detected and identified by the neural network in FIG. 11B.

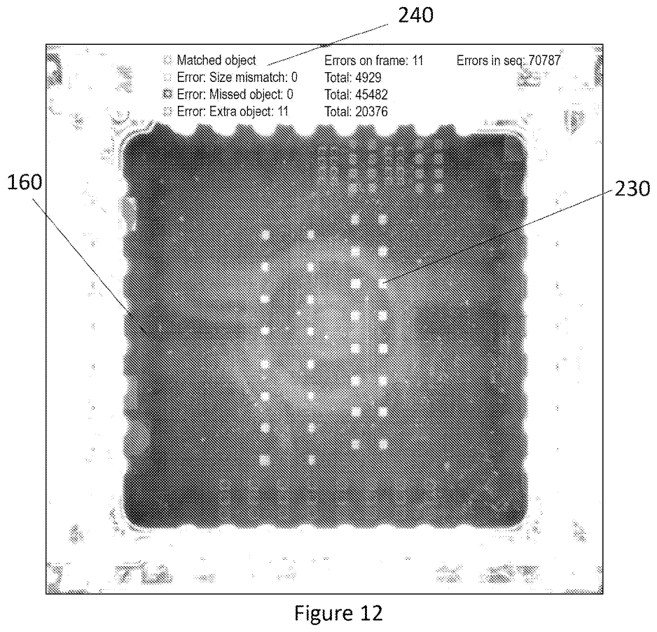

From the detected features of interest 230, a spatial distribution and shape of the features of interest 230 is determined. In this example, determining a spatial distribution and shape of features of interest 230 comprises generating bounding boxes around the features of interest 230. An example of this is shown in the image of FIG. 12, in which features of interest 230 have been bounded. Generating bounding boxes around the features of interest 230 provides a spatial distribution and shape suitable for direct comparison with the expected spatial distribution and shape 150 (i.e. the simulation of the experiment). As can be seen in the simulation 150 of FIG. 3, for example, features 170 typically take the form of squares and rectangles. Therefore, bounding boxes in the received image 160 of the digital microfluidic volume 200 will better correspond to the simulation 150, making it easier to determine if that image 160 of the digital microfluidic experiment 20 corresponds to what is expected at that point of the experiment 20 from the simulation 150. It will be appreciated that determining a spatial distribution and shape of features of interest 230 need not include the generation of bounding boxes, and that in some examples, the spatial distribution and shape of the detected features of interest without bounding boxes may be compared with the expected spatial distribution and shape 150 using other suitable techniques. For example, the comparison may be performed by calculating the centroids of the detected features of interest 230 and comparing the positions of the calculated centroids with the expected spatial distribution and shape 150.

Once the spatial distribution and shape of features of interest 230 has been determined, at step 130 of the method outlined by the flowchart 70 of FIG. 2, the distribution is compared with the expected spatial distribution and shape 150. This is done by mapping features of interest 230 in the determined distribution to corresponding features in the expected spatial distribution and shape 150.

The mapping of features of interest is performed as follows. For each detected feature of interest in the image, a bounding box is generated (if a bounding box generated as part of the spatial distribution and shape determination step, then this may be re-used). The centre point of the bounding box is located, and the position of the centre point in the image is calculated. The position of the calculated centre point is then compared to the centre point positions of the feature bounding boxes in the expected spatial distribution, and the centre-to-centre distance between the feature of interest and each expected feature is calculated. As described above, the expected spatial distribution is typically stored as a list of expected features and their respective characteristics including size, shape, and position. The position characteristic stored for each expected feature may be the centre point of the bounding box for that expected feature.

The calculated centre-to-centre distances between the detected feature of interest and each expected feature in the expected spatial distribution are assessed to determine if there is a “matched” feature in the expected distribution. In preferred embodiments, this is achieved by comparing the calculated centre-to-centre distances with a threshold distance. If a centre-to-centre distance is less than (or less than or equal to) the threshold distance, the detected feature of interest is assigned to the corresponding feature in the expected spatial distribution.

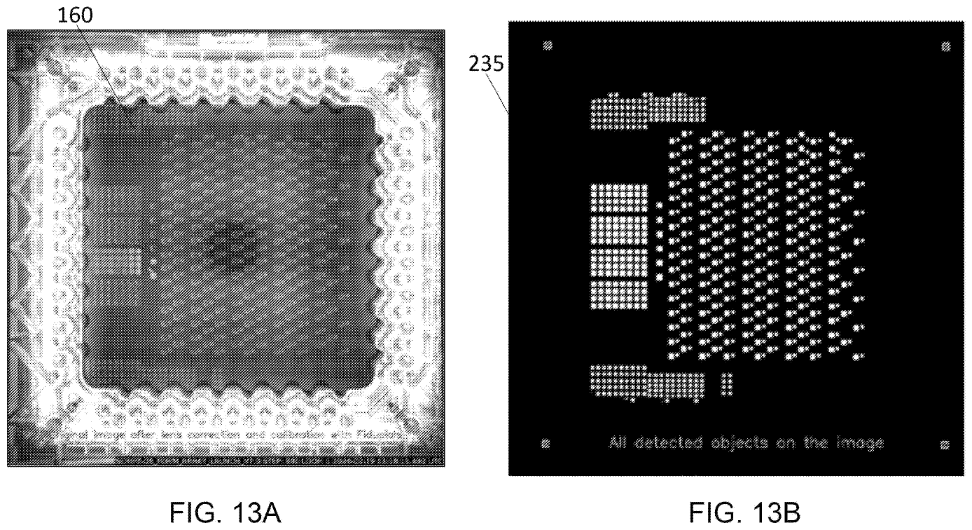

It will be appreciated that other suitable methods may be used to compare the detected spatial distribution with the expected spatial distribution, for example by overlaying a 2D binary mask of the detected spatial distribution over a 2D binary mask of the expected spatial distribution, and highlighting any discrepancies between the two distributions. FIGS. 13A and 13B respectively show an image of a digital microfluidic volume 160 and a corresponding 2D binary mask 235 of the detected spatial distribution. The image of the digital microfluidic volume has been corrected for lens distortion. Furthermore, a calibration transformation has been applied to the image using the fiducial markers, as discussed above.

Based on this comparison, at step 140, the presence or absence of erroneous features is detected based on the comparison. In general, an erroneous feature is one that results from a mismatch between the expected distribution 150 and the determined distribution from the image 160 of the experiment 20. In this example, if the size of a feature of interest 230 in the image 160 does not match what is expected, that feature of interest 230 is labelled erroneous. Furthermore, if a spatial position of a feature of interest 230 does not match what is expected, that feature of interest 230 is labelled erroneous. The same is true vice versa. Furthermore, correctly shaped droplets are square or rectangular in shape. An irregularly shaped feature of interest indicates an incorrect volume of fluid in the relevant droplet. Therefore, if the shape of a feature of interest does not match what is expected, the feature is labelled erroneous. The same is true vice versa. In other words, if a spatial position or shape of a feature 170 in the expected distribution 150 is not matched by the image 160, that feature of interest 230 is labelled erroneous. A ‘missing’ feature of interest 230 will also be labelled erroneous. In other words, if a feature 170 in the expected distribution 150 has no corresponding feature in the image 160, or vice versa.

In this example, detected erroneous features 230 are assigned a category. In the example shown in FIG. 12, a first category is a ‘size mismatch’ category. This category corresponds to the size of a feature of interest 230 not matching that of the corresponding feature 170 in the expected spatial distribution and shape 150. A second category is a ‘missed object’ category. This category corresponds to a feature 170 in the expected distribution 150 not being reciprocated in the image 160 of the experiment 20. A third category is a ‘extra object’ category. This category corresponds to an extra feature of interest 230 being detected in the image 160 that is not reciprocated by the expected spatial distribution and shape 150. In this example, the colour of the bounding box surrounding a feature of interest 230 indicates its category. This allows the operator of the experiment to easily identify on inspection what type of error has been detected. It will be appreciated that the invention need not be limited to the aforementioned categories, and any other categories may be assigned depending on the particular application.

In addition to detecting the presence of erroneous features, the method further comprises detecting the absence of erroneous features. In other words, features that are correct based on comparison with the expected spatial distribution and shape. A correct feature in this context is one that is not labelled erroneous. Specifically, a feature of interest 230 that matches the size, spatial position, and shape of a corresponding feature 170 in the expected spatial distribution and shape 150. Such features are also assigned a category. In this example, this is a fourth category ‘matched object’. An example of the process of detecting the presence/absence of erroneous features is set out below.

As described above, if the centre-to-centre distance between a detected feature and an expected feature in the expected spatial distribution is less than a threshold value, then detected feature is assigned to expected feature in the expected spatial distribution. The expected feature to which a detected feature is assigned may be referred to as the “corresponding” feature. If a detected feature of interest is assigned to a corresponding feature in the expected spatial distribution, then the characteristics of the detected feature and the corresponding feature are compared to determine if the feature is a “Match” or “Matched Object”.

For example, If a detected feature is assigned to an expected feature in the expected spatial distribution, the size (e.g. droplet size) of a detected feature may be compared to the size of the corresponding feature. If the difference between the sizes is within a size difference threshold, then the detected feature may be reported as a “Match”. If the difference between the sizes is not within a size difference threshold, then the detected feature is reported as a “Size Warning”.

It will be appreciated that other feature properties may be used in addition to, or in place of, the size to determine whether a detected feature is a “Match”.

If the none of the calculated centre-to-centre distances between the detected feature and the expected features in the expected spatial distribution are less than the threshold distance, then the detected feature may be reported as an “Extra Object” (extraneous feature).

After all of the detected features in the image have been analysed to detect erroneous features, the system determines whether any expected features in the expected spatial distribution have not been assigned to a detected feature in the image (or had a detected feature in the image assigned to them). If an expected feature in the expected spatial distribution has not been assigned a detected feature, or vice versa, the expected feature is labelled as a “Missing Feature”.

In alternative embodiments, the detection of erroneous features may be supplemented by additional feature labelling and characterisation that may be performed via the feature detection output of the machine learning models. Specifically, in some examples the models may be trained to automatically characterize erroneous features such as air bubbles, contaminants, and image artifacts, or perform measurements on the detected features of interest.

It will be appreciated that the detection of erroneous features via a comparison between a detected spatial distribution of features and an expected spatial distribution allows for a broad variety of potential errors to be detected. Compared to conventional techniques of the prior art, the present invention allows each characteristic of a particular detected feature to be compared with, and validated against, an expected value. Therefore errors related to position, shape, size, can be detected, along with errors relating to missing features and extraneous features, as set out below.

The method further comprises reporting the presence or absence of erroneous features at step 145 of the flowchart 70 of FIG. 2. In some examples, this comprises generating a labelled image, and outputting the labelled image to be displayed to a user via a display screen. The labelled image may, for example, correspond to the captured image of the digital microfluidic experiment captured by the imaging means, wherein each detected feature has been annotated according to its determined category. The annotation may comprise displaying bounding boxes around the detected features in a certain colour depending on the assigned category. For example, size mismatched features may be displayed with a bounding box of a first colour, missed object features may be displayed with a bounding box of a second colour, and extra object features may be displayed with a bounding box of a third colour. The missed object features may be displayed by placing a bounding box of the expected size at the expected location, based on the expected spatial distribution and shape 150, in the labelled image. Correct objects may be displayed with a bounding box of a fourth colour. As will be clear to the skilled person, errors can be reported without generating a labelled image. For example, in other embodiments, reporting the presence or absence of erroneous features comprises outputting a total number of erroneous features corresponding to each category. In some examples, the method further comprises acting to correct the erroneous features. The error correction may be as a result or user involvement and correction, or may be automated. The image processing can give users an idea of the potential errors and to provide a pass/fail measurement for the experiments, or may involve active error correction.

Particular embodiments may be beneficial when the droplet operations take place over many hours, for example greater than 2 hours. Certain operations may take place over for example 12 hours for complex patterns of mixing and incubation. The user does not wish to monitor the droplet handling operations for many hours. An automated system for tracking and alerting a user to failures of particular droplets is therefore advantageous, particularly if the error can be corrected automatically, for example by adding fresh reagent or repeating a droplet movement operation. Disclosed is a method involving analysing a series of two or more images of a digital microfluidic volume taken during the course of an experiment and reporting information from all images from the series to determine whether each feature of interest is correct at the end of the digital microfluidic experiment.

The image detection can identify that while a droplet is present in the image, the contents of the droplet cannot be trusted. Image tracking knows this because it has reviewed multiple other images in the droplets ‘history’ and found that it misbehaved in one of these historical images. Therefore whilst the correctly sized droplet is present in the correct location, its contents may be suspect due to previous errors in movement or droplet collisions. For example an experiment may capture 10 frames throughout the duration of the experiment. If for example in frame 2 of these 10, there is an error, and although there are no more errors anywhere else throughout the run, the method herein flags that reaction zone/droplet as a fail, because in one of the frames there was an issue with at least ONE of its components.

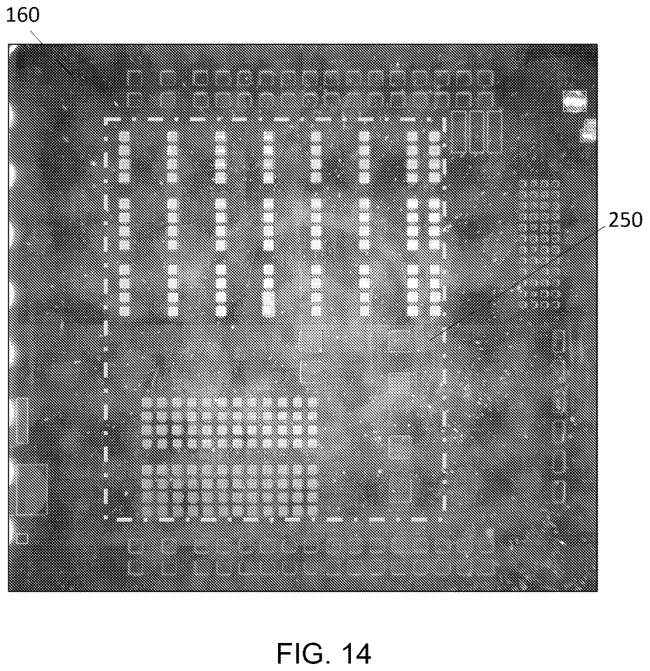

In preferred embodiments, the method may refrain from labelling and/or reporting a detected feature as an erroneous feature if the detected feature falls within an “ignore area” of the image. The “ignore area” may be a predetermined region, or regions, of the image that are not relevant to the experimental outcome. For example, an “ignore area” of the image may be configured to include the boundary areas of the image that do not form part of the experimental volume. In other words, they do not form part of the “active” experimental area. The above described method is repeated for a sequence of images 160 taken as the experiment 20 progresses. In other words, images 160 are taken periodically as the experiment 20 is carried out. Images are taken with commands from the script which is unique to each workflow. For example in some scripts it is at every 10 steps, (each step being approximately 1.5 s) and in some it is every 14, etc. In some examples the sequence of images 160 may represent frames of video data captured by the imaging means. Errors are detected in using the above described method in real time based on the captured images 160. The method further comprises annotating the received images 160 based on the detected features 230 and their assigned categories. In the example shown in FIG. 12, the annotation 240 comprises colour coded bounding boxes based on category. The annotation 240 further comprises a list of possible categories and a corresponding numerical value quantifying how many of such errors have been detected. The annotation 240 further comprises total error values based on the sequence of images 160 of the digital microfluidic experiment 20 as it progresses. Such annotation 240 is useful for the experiment operator. On inspection, they are able to obtain an overview of how successful the experiment 20 is in real time. They are able to obtain a snapshot view of whether, at a point in time, the experiment 20 corresponds to what is expected based on a single image 160. They are further able to obtain a cumulative idea of the experiment's 20 success over time based on the sequence of images 160. Some regions of the experiment may be of more interest to the operator than others. For example, errors in said region may pose more of a problem than errors in other regions of the experiment. The operator can, therefore, select a region of interest 250 to focus error detection on, as shown in FIG. 14.

### Errors in the System May Include One or More of the Following:

### Missing Droplets

Droplets may fail to be dispensed into the device or from a reservoir on the device. Active feedback may allow the dispense to be repeated to correct the error. Alternatively droplets may be dispensed but fail to move. Such droplets in the incorrect place may be tracked and either corrected using dynamic movement scripts or may be tracked knowing where they are in reality vs where they are supposed to be.

### Incorrectly Sized Droplets

Droplets may be dispensed by be either too large or too small in volume. Such droplets may or may not be moved correctly. If the volume is known and measured, then analysis may correct the fluorescent signal based on the droplet size. Such mis-sized droplets may be apparent as having a different shape to the correct droplets.

### Droplet Collision

Droplets which do not move correctly may collide with other droplets. Such droplets can be ignored for further consideration as they will contain the incorrect reagents. Such droplets are typically oversized and in the wrong location in the relation to the correct droplets.

### Failure to Move

Droplets may in some circumstances fail to move when driven. If the error is spotted, the droplet move command can be repeated in order to drive the particular droplets that are not in the correct location. Optionally a second different driving script may be used to move particular droplets which failed to move using a first driving script.

### Air Bubbles

The introduction of bubbles may occur either from the atmosphere or from damage on the device causing electrolysis or degassing. Air bubbles can either be removed, for example by moving the filler fluid, or may be ignored if they are apparent on the device. Air bubbles are typically not the same shape as droplets so can be tracked.

### Extraneous Features

Features such as dust on the device may appear as droplets or may scatter light so as to obscure the presence of droplets. Such features or visual distortions, which typically do not move, can be removed using imaging processing techniques such the droplets can be tracked.

### No Errors

A experiment may be executed with no observed errors, and such result reported to the user. When execution of an experiment is completed, the method provides as output, for each image captured using the imaging means, an indication of any detected errors in each image. The output may be in the form of a list of objects and errors including matching, extra, missing, and mis-sized objects for each input image, and labels indicating whether the errors were detected in any ignore areas. The indication may be in the form of a JSON file, or another suitable file type.

Based on the output, the method may assign an overall score for the executed experiment. An example of how an overall score for the experiment may be calculated is as follows, however it will be appreciated that the score may be calculated using an alternative method. For example, if the output indicates that no errors were detected in any of the images captured using the imaging means during the experiment, then the method may assign a “PASS” score to the experiment. If the output indicates that a number of errors greater than a threshold number of errors, were detected in a number of images greater than a threshold number of images, the method may assign a “FAIL” score to the experiment. If the output indicates that a number of errors less than a threshold number of errors, were detected in one or more images, the method may assign a “Pass with Concession” score to the experiment. If the output indicates that a number of errors greater than a threshold number of errors, were detected in a number of images less than a threshold number of images, the method may assign a “Pass with Concession” score to the experiment. The overall score for the experiment may be reported to the user based on the analysis of a series of images captured during the experiment.

### Applications

Provided herein are methods for improved expression of proteins in cell-free systems and purification thereof. Expression of proteins may take many hours, for example greater than 2 hours or greater than 4 hours. The process may be initiated by mixing a nucleic acid template with reagents for cell-free protein expression. Droplets are typically moved during the expression process to ensure mixing, A detector reagent may be added after expression to detect if a protein with a tag has been produced. The droplet operation process may fail at any point during the expression process, and a user does not want to have to monitor the entire process to ensure that the correct results are obtained. An automated system for tracking and alerting a user to failures of particularly droplets is therefore advantageous, particular if the error can be corrected automatically, for example by adding fresh reagent or repeating a droplet movement operation.

Proteins are biological macromolecules that maintain the structural and functional integrity of the cell, and many diseases are associated with protein malfunction. Protein purification is a fundamental step for analysing individual proteins and protein complexes and identifying interactions with other proteins, DNA or RNA. A variety of protein purification strategies exist to address desired scale, throughput and downstream applications. However, protein production can be challenging for many reasons. One major challenge is finding a suitable expression system, for example sourced from mammalian, bacterial, fungal, or plant cells. This can take months of work.

Cell-free protein synthesis (CFPS), also known as coupled or uncoupled in-vitro transcription and translation, is the production of peptides or proteins using biological machinery in a cell-free system, that is, without the use of living cells. The CFPS environment is not constrained within a cell wall or limited by conditions necessary to maintain cell viability, and enables the rapid production of any desired protein from a nucleic acid template, usually plasmid DNA or RNA from an in-vitro transcription. CFPS has been known for decades, and many commercial systems are available. Cell-free protein synthesis encompasses systems based on crude lysate (Cold Spring Harb Perspect Biol. 2016 December; 8(12): a023853) and systems based on reconstituted, purified molecular reagents, such as the PURE system for protein production (Methods Mol Biol. 2014; 1118: 275-284). CFPS requires significant concentrations of biomacromolecules, including DNA, RNA, proteins, polysaccharides, molecular crowding agents, and more (Febs Letters 2013, 2, 58, 261-268).

Split detectors such as split green fluorescent protein (GFP) systems are known for use in protein expression. A protein of interest having a GFP subcomponent can be detected by complementing with a detector species having the remainder of the GFP. Cabantous and Waldo describe such a system (In-vivo and in-vitro protein solubility assays using split GFP (NATURE METHODS|VOL.3 NO.10|OCTOBER 2006|845). The system described relies on expression in cells, from which the proteins of interest are lysed and then exposed to the detector in order to measure the level of expression.

WO2022/038353 describes cell-free expression of a protein having a GFP tag in the presence of a GFP detector species to measure signal as expression progresses.

Many proteins are expressed containing endogenous solubility factors. However factors such as for example maltose binding protein (MBP), Small Ubiquitin-like Modifier (SUMO), Glutathione S-transferase (GST) or thioredoxin (TRX) have a substantial size and may have undesirable effects on the proteins of interest to which they are attached. Additionally expression of large solubility elements uses the reagent resources during expression. Such sequences also take time to fold before acting at solubility enhancers, hence synthesising proteins of interest with solubility enhancing sequences attached thereto is far from ideal.

To date protein purification and analysis typically requires complex analysis techniques involving electrophoresis or requiring purified proteins. The inventors herein have developed simplified protein analysis and purification methods allowing multiple ways of characterising expressed proteins in their crude form.

One of the current challenges for cell-free protein synthesis is to increase the soluble yield of the expressed and purified proteins and to avoid aggregation or insolubility.

Disclosed is a method that relies on parallel handling of volumes of fluid. Disclosed is a method for synthesising, characterising and purifying one or more proteins having detection tags and binding tags.

The POI may be expressed with a binding sequence which binds to a detector moiety. In which case a further droplet containing a detector moiety may be added to the droplet containing the POI to create an detectable signal in the combined droplet. The binding sequences may contain four or more amino acids. The binding sequences may contain 4-30 amino acids.

The detector moiety may be a protein. The detector moiety may comprise a component of a fluorescent protein such as for example sfGFP or ccGFP.

The expressed protein may contain a sequence acting as a solubility enhancer, for example selected from:

| | |
|---|---|
| Glutathione S-Transferase | GST |
| Small Ubiquitin-like Modifier | SUMO |
| Maltose Binding Protein | MBP |
| Fasciola hepatica 8 kDa antigen | FH8 |
| Thioredoxin | TRX |
| Solubility Enhancing Ubiquitous Tag | SNUT |
| Seventeen kilodalton protein | SKP |
| Monomeric bacteriophage T7 orc protein | MOCR |
| E coli secreted protein A | ESPA |
| N-utilization substance | NusA |
| IgG domain B1 of Protein G | GB1 |
| IgG repeat domain ZZ of Protein A | ZZ |
| Mutated dehalogenase | HaloTag |
| Phage T7 protein kinase | T7PK |
| E. coli trypsin inhibitor | Ecotin |
| Calcium-binding protein | CaBP |
| Stress-response arsenate reductase | ArsC |
| N-terminal fragment of translation initiation factor IF2 | IF2-domain 1 |
| Stress-response protein | RpoA |
| Stress-response protein | SlyD |
| Stress-response protein | Tsf |
| Stress-response protein | RpoS |
| Stress-response protein | PotD |
| Stress-response protein | Crr |
| E. coli acidic protein | msyB |
| E. coli acidic protein | yjgD |
| E. coli acidic protein | rpoD |

The detection tag may be one component of a fluorescent protein and the detector reagent a complementary portion of the fluorescent protein. The fluorescent protein could include sfGFP, GFP, eGFP, deGFP, frGFP, eYFP, eEFP, eCFP, Citrine, Venus, Cerulean, Dronpa, DsRED, mKate, mCherry, mRFP, FAST, SmURFP, miRFP670nano. For example the tag may be GFP₁₁ and the detector GFP₁₋₁₀. The tag may be one component of sfCherry. The tag may be sfCherry~₁ and the detector sfCherry₁₋₁₀. The tag may be CFAST₁₁ or CFAST₁₀ and the detector NFAST in the presence of a hydroxybenzylidene rhodanine analog.

The tag may be ccGFP₁₁ and the detector ccGFP₁₋₁₀.

The expression may be performed using cell-free protein synthesis reagents derived from whole cell extracts. The expression may be performed using cell-free protein synthesis reagents derived from reconstituted systems comprising assembled components for transcription and translation in a system of purified recombinant elements (PURE).

Disclosed is a method for expressing proteins in droplets on a digital microfluidic device having a two-dimensional array of planar microelectrodes wherein the proteins have a ccGFP₁₁ peptide amino sequence tag, wherein a portion of the droplets contain ccGFP₁₋₁₀ during the expression process and a further portion have ccGFP₁₋₁₀ added after expression and comparing the level of ccGFP₁₋₁₁ signal from the droplets.

The binding moiety for purification may contain four or more amino acids. The binding sequences may contain 4-30 amino acids. The binding moiety may be selected from:

Alfa-tag
(SRLEEELRRRLTE)
Avi-tag
(GLNDIFEAQKIEWHE)
C-tag
(EPEA)
Calmodulin-tag
(KRRWKKNFIAVSAANRFKKISSSGAL)
Dogtag
(DIPATYEFTDGKHYITNEPIPPK)
E-tag
(GAPVPYPDPLEPR)
FLAG
(DYKDDDDK)
G4T
(EELLSKNYHLENEVARLKK)
HA
(YPYDVPDYA)
His
(HHHHHH)
Isopeptag
(TDKDMTITFTNKKDAE)
Ianthanide binding tag (LBT)
(FIDTNNDGWIEGDELLLEEG)
Myc
(EQKLISEEDL)
NE-Tag
(TKENPRSNQEESYDDNES)
Poly Glutamate-tag
(EEEEEEE)
Poly Arginine-tag
(RRRRRRR)
Rho1D4-tag
(TETSQVAPA)
SBP-tag
(MDEKTTGWRGGHVVEGLAGELEQLRARLEHHPQGQREP)
Sdytag
(DPIVMIDNDKPIT)
SH3
(STVPVAPPRRRRG)
SNAC
(GSHHW)
Snooptag
(KLGDIEFIKVNK)
Softag 1
(SLAELLNAGLGGS)
Softag 3
(TQDPSRVG)
Spot-tag
(PDRVRAVSHWSS)
Spytag
(AHIVMVDAYKPTK)
S-tag
(KETAAAKFERQHMDS)
Strep-tag
(WSHPQFEK)
Strep-tag II
(AWAHPQPGG)
T7tag
(MASMTGGQQMG)
TC-tag
(EVHTNQDPLD)
Ty-tag
(CCPGCC)
VSV-tag
(YTDIEMNRLGK)
Xpress-tag
(DLYDDDDK)

The method may be performed on different sequences in parallel. The method may use at least 8 different nucleic acid templates, which may be screened against at least 4 different expression reagents on the same device. The device may be capable of handling many droplets in parallel, for example the device may separately manipulate at least 192 droplets. The expression comparison and purification screen can identify the optimal conditions for expression and purification of the desired protein in its most soluble and stable form.

The POI may be expressed with a binding sequence which binds to a detector moiety. In which case a further droplet containing a detector moiety may be added to the droplet containing the POI to create an detectable signal in the combined droplet.

Embodiments of the present invention have been described with particular reference to the examples illustrated. However, it will be appreciated that variations and modifications may be made to the examples described within the scope of the present invention.

As will be appreciated by one of skill in the art, the invention described herein may be embodied in whole or in part as a method, a data processing system, or a computer program product including computer readable instructions. Accordingly, the invention may take the form of an entirely hardware embodiment or an embodiment combining software, hardware and any other suitable approach or apparatus.

The computer readable program instructions may be stored on a non-transitory, tangible computer readable medium. The computer readable storage medium may include one or more of an electronic storage device, a magnetic storage device, an optical storage device, an electromagnetic storage device, a semiconductor storage device, a portable computer disk, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk.

Exemplary embodiments of the invention may be implemented as a circuit board which may include a CPU, a bus, RAM, flash memory, one or more ports for operation of connected 1/O apparatus such as printers, display, keypads, sensors and cameras, ROM, a communications sub-system such as a modem, and communications media.

As will be appreciated by one of skill in the art, the term “processing means” may correspond to any suitable processing device. For example, the processing means may be any of a computer processor, graphics processor, programmable logic device, microprocessor, or any other suitable device. It will be appreciated that the processing means may comprise a plurality of processing devices, and may be a combination of different processing devices such as those described above.

Example of image process of unseen images using a model trained on seen images. 540 images were used to train the processing module, which was them used to process 140 unseen images which have 47460 objects in total (an average of 339 droplets per image).

Data from the unseen images is shown in the table below:

| Total | Detected | Missed | False | Object | | F1 | False Positive |
|---|---|---|---|---|---|---|---|
| Objects | Objects | Objects | Positives | Accuracy | Precision | Score | Rate |
| 47460 | 47328 | 132 | 149 | 99.72% | 99.69% | 99.70% | 0.31% |

The model can thus be used to detect the presence or absence of the correct droplets and provide an automated report to a user.

### Droplet Tracking

Error detection due to previous droplet movements. An example from the eProtein Discovery system where protein expression droplets are tracked (192 droplets for 10 hours of expression, merging with detector and 5 hours of incubation for detection), then 32 purification conditions and controls. Experiments are tracked over greater than 24 hours in duration for expression and purification conditions. Results from experiments are analysed as a single final output file that analyses a number of frames at selected points during the experimental process. For any row in the file that you see a FAIL in the QC column, the process tells you where exactly it detected an error and why it is failing that row. In the tables shown below, 2 of the 30 purification zones are marked as failed despite droplets being detected in the final image frame. The missing droplets occurred in earlier images than the final frame and were detected as errors in the final results. The automatic alerting of errors from previous frames aids processing of results as the need to manually review the whole experiment is removed.

| Results | |
|---|---|
| Summary | Status |
| Final | FAIL |
| Purification | |
| Quality | |
| Control | |

| | | | | | | ROIs | ROIs |
|---|---|---|---|---|---|---|---|
| | | | Pass | Valid | | with | with |
| | Expected | Valid | Criteria | rate | Status | Error | Warning |
| Number of | 10 | 10 | 100% | 100.00% | PASS | [ ] | [36] |
| Controls: | | | | | | | |
| Number of | 30 | 28 | 95% | 93.33% | FAIL | [16, | [ ] |
| Purification | | | | | | 30] | |
| Droplets | | | | | | | |

| region | | | | |
|---|---|---|---|---|
| ID | reactionZoneName | type | QC | errors |
| 1 | A12_CFB01_d3_and_A3_eGene03_ | Purification | PASS | |
| | d7_hit_eluted_and_ndet | | | |
| 2 | A12_CFB01_d14_and_A7_ | Purification | PASS | |
| | eGene07_d7_hit_eluted_and_ndet | | | |
| 3 | A12_CFB01_d16_and_A8_ | Purification | PASS | |
| | eGene08_d7_hit_eluted_and_ndet | | | |
| 4 | A12_CFB01_d15_and_C1_ | Purification | PASS | |
| | eGene17_d1_hit_eluted_and_ndet | | | |
| 5 | A12_CFB01_d13_and_B7_eGene15_ | Purification | PASS | |
| | d7_hit_eluted_and_ndet | | | |
| 6 | A12_CFB01_d17_and_B8_eGene16_ | Purification | PASS | |
| | d7_hit_eluted_and_ndet | | | |
| 7 | B12_CFB02_d4_and_A2_eGene02_ | Purification | PASS | |
| | d8_hit_eluted_and_ndet | | | |
| 8 | B12_CFB02_d10_and_A4_eGene04_ | Purification | PASS | |
| | d8_hit_eluted_and_ndet | | | |
| 9 | B12_CFB02_d12_and_A5_eGene05_ | Purification | PASS | |
| | d8_hit_eluted_and_ndet | | | |
| 10 | B12_CFB02_d11_and_A6_eGene06_ | Purification | PASS | |
| | d8_hit_eluted_and_ndet | | | |
| 11 | B12_CFB02_d14_and_A7_eGene07_ | Purification | PASS | |
| | d8_hit_eluted_and_ndet | | | |
| 12 | B12_CFB02_d16_and_A8_eGene08_ | Purification | PASS | |
| | d8_hit_eluted_and_ndet | | | |
| 13 | B12_CFB02_d15_and_C1_eGene17_ | Purification | PASS | |
| | d2_hit_eluted_and_ndet | | | |
| 14 | B12_CFB02_d22_and_C3_eGene19_ | Purification | PASS | |
| | d2_hit_eluted_and_ndet | | | |
| 15 | B12_CFB02_d24_and_C5_eGene21_ | Purification | PASS | |
| | d2_hit_eluted_and_ndet | | | |
| 16 | B12_CFB02_d1_and_B1_eGene09_ | Purification | FAIL | [{‘SCRIPT08_A10_ |
| | d8_hit_eluted_and_ndet | | | NDET01_part2_d28’: |
| | | | | [[{‘errorType’: |
| | | | | MISSED’, ‘errorFrame’: |
| | | | | ‘expression_004_001_ |
| | | | | 00840’}]]}] |
| 17 | B12_CFB02_d7_and_B5_eGene13_ | Purification | PASS | |
| | d8_hit_eluted_and_ndet | | | |
| 18 | B12_CFB02_d8_and_B6_eGene14_ | Purification | PASS | |
| | d8_hit_eluted_and_ndet | | | |
| 19 | B12_CFB02_d13_and_B7_eGene15_ | Purification | PASS | |
| | d8_hit_eluted_and_ndet | | | |
| 20 | B12_CFB02_d17_and_B8_eGene16_ | Purification | PASS | |
| | d8_hit_eluted_and_ndet | | | |
| 21 | B12_CFB02_d18_and_C2_eGene18_ | Purification | PASS | |
| | d2_hit_eluted_and_ndet | | | |
| 22 | B12_CFB02_d21_and_C4_eGene20_ | Purification | PASS | |
| | d2_hit_eluted_and_ndet | | | |
| 23 | B12_CFB02_d19_and_C6_eGene22_ | Purification | PASS | |
| | d2_hit_eluted_and_ndet | | | |
| 24 | C12_CFB03_d2_and_A1_eGene01_ | Purification | PASS | |
| | d5_hit_eluted_and_ndet | | | |
| 25 | C12_CFB03_d4_and_A2_eGene02_ | Purification | PASS | |
| | d5_hit_eluted_and_ndet | | | |
| 26 | C12_CFB03_d3_and_A3_eGene03_ | Purification | PASS | |
| | d5_hit_eluted_and_ndet | | | |
| 27 | C12_CFB03_d16_and_A8_eGene08_ | Purification | PASS | |
| | d5_hit_eluted_and_ndet | | | |
| 28 | C12_CFB03_d15_and_C1_eGene17_ | Purification | PASS | |
| | d3_hit_eluted_and_ndet | | | |
| 29 | C12_CFB03_d28_and_H5_CTRL_ | Control | PASS | |
| | Purification_d1_hit_eluted_and_ndet | | | |
| 30 | C12_CFB03_d1_and_B1_eGene09_ | Purification | FAIL | [{‘SCRIPT18_C12_ |
| | d5_hit_eluted_and_ndet | | | CFB03_d1_and_B1_eGene |
| | | | | 09_d5_purify’: |
| | | | | [{‘errorType’: |
| | | | | ‘MISSED’, ‘errorFrame’: |
| | | | | ‘purification_001_001_ |
| | | | | 02716’}]]}] |
| 31 | C12_CFB03_d18_and_C2_eGene18_ | Purification | PASS | |
| | d3_hit_eluted_and_ndet | | | |
| 32 | G12_CFB07_d28_and_H7_CTRL_ | Control | PASS | |
| | Workflow_d1_hit_eluted_and_ndet | | | |
| 33 | H2_CTRL_Negative_Purification_d2_ | Control | PASS | |
| | and_A10_NDET01_part1_d1 | | | |
| 34 | H2_CTRL_Negative_Purification_d1_ | Control | PASS | |
| | and_A10_NDET01_part1_d2 | | | |
| 35 | Blank Buffer | Control | PASS | |
| 36 | Blank Buffer | Control | Pass | [{‘SCRIPT18_H3_STD_ |
| | | | with | 0_d1’: [{‘errorType’: |
| | | | con- | ‘SIZE’, ‘errorFrame’: |
| | | | cession | ‘purification_001_001_ |
| | | | | 02716’}]]}, |
| | | | | {‘SCRIPT20_H3_STD_ |
| | | | | 0_d1’: [{‘errorType’: |
| | | | | ‘SIZE’, ‘errorFrame’: |
| | | | | ‘purification_003_001_ |
| | | | | 00112’}]]}, |
| | | | | {‘SCRIPT21_H3_STD_ |
| | | | | 0_d1’: [{‘errorType’: |
| | | | | ‘SIZE’, ‘errorFrame’: |
| | | | | ‘purification_004_001_ |
| | | | | 00112’}]]}, |
| | | | | {‘SCRIPT22_H3_STD_ |
| | | | | 0_d1’: [{‘errorType’: |
| | | | | ‘SIZE’, ‘errorFrame’: |
| | | | | ‘purification_005_001_ |
| | | | | 00112’}]]}, |
| | | | | {‘SCRIPT23_H3_STD_ |
| | | | | 0_d1’: [{‘errorType’: |
| | | | | ‘SIZE’, ‘errorFrame’: |
| | | | | ‘purification_006_001_ |
| | | | | 00118’}]]}] |
| 37 | Universal Control | Control | PASS | |
| 38 | Universal Control | Control | PASS | |
| 39 | E12_CFB05_d28_and_H6_CTRL_ | Control | PASS | |
| | Complementation_d1_purify_and_A10_ | | | |
| | NDET01_part1_d13 | | | |
| 40 | F12_CFB06_d28_and_H6_CTRL_ | Control | PASS | |
| | Complementation_d2_purify_and_A10_ | | | |
| | NDET01_part1_d14 | | | |

1. A method for detecting and reporting errors in a digital microfluidic experiment, the method comprising:
receiving image data comprising an image of a digital microfluidic volume;
detecting one or more features of interest corresponding to droplets of fluid in the digital microfluidic volume in the image using a trained convolutional neural network;
determining a spatial distribution and shape of the detected droplets of fluid;
receiving an expected spatial distribution and shape of features;
spatially aligning the received image and the expected spatial distribution and shape of features by detecting a plurality of fiducial markers in the received image; and applying a transformation to the received image based on the detected fiducial markers;
comparing the spatially aligned image with the corresponding features in the expected spatial distribution;
determining the presence or absence of one or more erroneous droplets based on the comparison; and
reporting the presence or absence of one or more erroneous droplets.

2. (canceled)

3. (canceled)

4. The method of claim 1 further comprising training a convolutional neural network to detect features of interest using images of digital microfluidic volumes in which features of interest have been segmented.

5. The method of claim 1 further comprising prior to the detecting one or more features of interest, adjusting the received image.

6. The method of claim 5, wherein adjusting the received image comprises applying a transformation to the received image to correct a lens distortion, the applying a transformation comprising applying a lens distortion matrix.

7. The method of claim 6 further comprising prior to the transforming the received image, calculating the lens distortion matrix.

8. The method of claim 5, wherein adjusting the received image comprises enhancing the visibility of the features of interest in the image of the digital microfluidic volume.

9. (canceled)

10. (canceled)

11. The method of claim 1, wherein detecting one or more erroneous features of interest comprises determining that a position or shape of a detected feature of interest in the received image does not match a position or shape of a corresponding feature of interest the expected spatial distribution, or vice versa.

12. The method of claim 1, wherein detecting one or more erroneous features of interest comprises determining that the size and shape of a detected feature of interest in the received image does not match a size and shape of a corresponding feature of interest in the expected spatial distribution.

13. The method of claim 1, wherein detecting one or more erroneous features of interest comprises detecting a feature of interest in the received image with no corresponding feature of interest in the expected spatial distribution, or vice versa.

14. The method of claim 1, wherein the expected spatial distribution corresponds to a simulation of the digital microfluidic volume and no errors are reported.

15. The method of claim 1, wherein determining the spatial distribution of the detected one or more features of interest comprises generating bounding boxes around each of the detected one or more features of interest, and comparing the spatial distribution of the detected one or more features of interest with the expected spatial distribution comprises comparing a spatial distribution of the generated bounding boxes with the expected spatial distribution.

16. The method of claim 1, further comprising assigning a category to a detected erroneous feature of interest based on the comparison of the determined spatial distribution of the detected one or more features of interest with the received expected spatial distribution of features, and the category comprises a size mismatch category, and/or one of: a missed object category and an extra object category.

17. The method of claim 1, further comprising annotating the received image based on the detected one or more erroneous features of interest and acting to correct the erroneous features.

18-21. (canceled)

22. The method of claim 1, wherein the correctly shaped droplets are square or rectangular in shape and the incorrectly shaped droplets are irregular in shape, indicating an incorrect volume of fluid in the droplets.

23-25. (canceled)
